(19) United States
(12) Patent Application Publication
(10) Pub. No.: US 2010/0234692 A1
(43) Pub. Date: Sep. 16, 2010

(54) SYSTEM FOR HEALTH MANAGEMENT USED IN VEHICLE AND METHOD THEREOF

(76) Inventors: Bo-Jau Kuo, Taipei City (TW); Ching-Hsiu Yang, Taipei City (TW)

Correspondence Address: Muncy, Geissler, Olds & Lowe, PLLC, 4000 Legato Road, Suite 310, FAIRFAX, VA, 22033, US

(21) Appl. No.: 12/403,614

(22) Filed: Mar. 13, 2009

(51) Int. Cl. A61B 5/00

(52) U.S. Cl. 600/300

(57) ABSTRACT

This invention discloses a system of health management used in vehicle and a method thereof, the system comprising a host, a driving health monitoring center, a wireless transmission system, a vehicle speed monitoring device, a vehicle position detecting device, an alarm device, a bio-signal sensor, and a recording device. The bio-signal sensor detects a body information and the vehicle speed detecting device detects a acceleration/deceleration information to analyze the driving quality, and the vehicle position detecting device detects a latitude/longitude information and transmit them to the host. The recording device stores the information from the host, and transmits to the center to analyze, or presses the emergency button to respond to the host and the center, and then the center feedbacks to the host to control the telephone equipment, the alarm device, the low-voltage defibrillator or the high-voltage defibrillator to communicate, remind or rescue by the wireless transmission system.

### BACKGROUND OF THE INVENTION

1. Field of the Invention

The present invention relates to a system for health management used in vehicle and method thereof, and more particularly, to health and driving quality control for the drivers and demanded passengers.

2. Description of the Related Art

Everyone has the experience of driving or going by car in recent life, and most people have to drive and go by car every day. A person to work by driving or by car everyday must spend at least ten minutes seating in vehicle. Because it is a blind zone of every medical treatment when driving or by car, to monitor the driver's or the passenger's health status is good for health management besides saving time.

Especially, the accident has risen to the one of top 10 death reasons, and the ratio of the traffic accident is higher. The reasons for occurring traffic accidents are mostly resulted from the driver's sleep or illness, so people try to find out a method for pre-warning or preventing from sleeping or being ill.

Recently, the bio-signal is constructed on conventional wired transmission technology so long. Patients must be stuck several electrodes connected to an amplifier with a conducting wire, and then to process the analog/digital converting and digital signal processing. It is inconvenient in use and hard to apply on the vehicle for warning because there are many wires on patients' body and limits the patients' movement. Although some suppliers have developed several wireless systems, most devices have only single one function and cannot balance demands in many aspects. Most recorders cannot monitor the patient's health status at any time, and lose the pre-warning function in recording immediately. It is hard to be used in sleep warning or immediate health information.

### SUMMARY OF THE INVENTION

In view of the problems of prior arts, the present invention provides a system for health management used in vehicle and method thereof to be not only capable of transmitting health information to appointed locations or hospitals for emergency rescue when the driver and passenger are driving or seating in the car, but also capable of sending or feedbacking a sleep message to a roused system, and to understand the message of the passenger's comfort quality for the driver to construct a system of health management used in vehicle. In addition, respective vehicle centers can control driver's health and driving quality based on all relative information by far-distance wireless transmission and record.

To achieve the above object, the present invention provides a system of health management used in vehicle and a method to construct the system, the system comprising an electrocardiography collecting and feedback system, a sleep detecting and preventing system, a driving quality monitoring system and a blood oxygen and heartbeat detecting system, wherein the electrocardiography collecting and feedback system, the sleep detecting and preventing system, the driving quality monitoring system and the blood oxygen and heartbeat detecting system are including a host, a driving health monitoring center, at least one wireless transmission system, at least one vehicle speed monitoring device, at least one vehicle position detecting device, at least one alarm device, at least one bio-signal sensor, and at least one recording device. A body information of an organism detected by the bio-signal sensor and a latitude/longitude information of a vehicle received by the vehicle position detecting device are transmitted to the host, the host controls the alarm device so as to feedback to the driver or the passenger, and all information is recorded by recording device and is transmitted to the driving health monitoring center by the wireless transmission system for health management or emergency rescue. In addition, the vehicle speed monitoring device detects the velocity information of a vehicle and transmits to the host, the host calculates the velocity information to monitor the acceleration/deceleration status of the vehicle and controls the alarm device to feedback to the driver or the passenger automatically, and all information is recorded by recording device and is transmitted to the driving health monitoring center by the wireless transmission system for evaluating the driving quality and the variation of the steering wheel and the gas pedal.

Besides the above efficiency, the system of health management used in vehicle and the method thereof of the present invention further comprise an emergency button, a telephone equipment, a low-voltage defibrillator and a high-voltage defibrillator, so as to provide emergency rescue manually, communicate with rescue division bilaterally, freshen up and rescue.

### BRIEF DESCRIPTION OF THE DRAWINGS

Other objects, advantages, and novel features of the invention will become more apparent from the following detailed description when taken in conjunction with the accompanying drawing:

FIG. 1 is a schematic drawing of an electrocardiography collecting and feedback system according to the present invention.

FIG. 2 is a schematic drawing of a sleep detecting and preventing system according to the present invention.

FIG. 3 is a schematic drawing of a driving quality monitoring system according to the present invention.

FIG. 4 is a schematic drawing of a blood oxygen and heartbeat detecting system according to the present invention.

FIG. 5 is a schematic drawing of the electrocardiography detecting device according to the present invention.

FIG. 6 is a schematic drawing of a system for health management used in vehicle according to the present invention.

FIG. 7 is a process schematic drawing of a method for health management used in vehicle according to the present invention.

### DETAILED DESCRIPTION OF THE INVENTION

Although the invention has been explained in relation to several preferred embodiments, the accompanying drawings and the following detailed description are the preferred embodiment of the present invention. It is to be understood that the following disclosed descriptions will be examples of present invention, and will not limit the present invention into the drawings and the special embodiment.

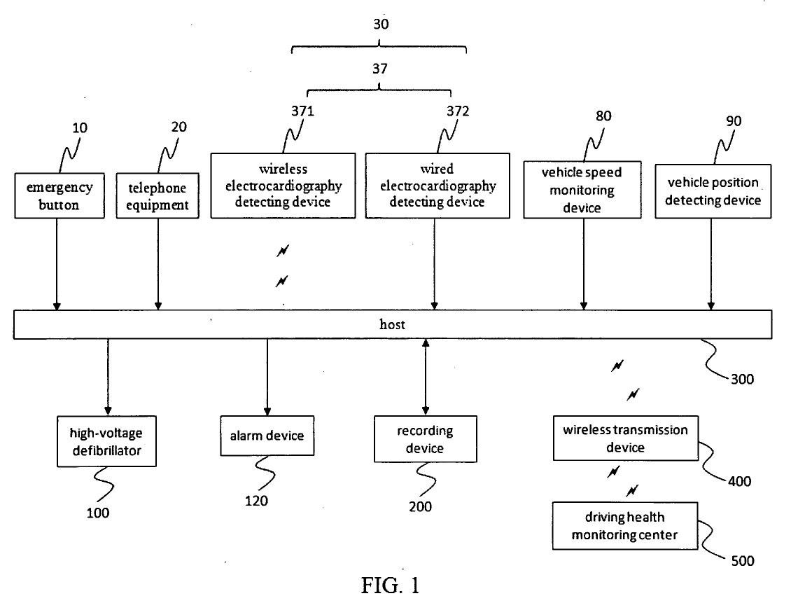

FIG. 1 is a schematic drawing of an electrocardiography collecting and feedback system according to the present invention. The electrocardiography collecting and feedback system 1 comprises a host 300, a driving health monitoring center 500, at least one wireless transmission system 400, at least one vehicle speed monitoring device 80, at least one vehicle position detecting device 90, at least one bio-signal sensor 30, at least one recording device 200, an emergency button 10, a telephone equipment 20, a high-voltage defibrillator 100 and at least one alarm device 120.

The driving health monitoring center 500 processes and responds a data of the host 300. The wireless transmission system 400 transmits the data between the host 300 and the driving health monitoring center 500. The vehicle speed monitoring device 80 detects the velocity information of a vehicle and transmits the velocity information to the host 300. The vehicle position detecting device 90 detects the latitude/longitude information of the vehicle and transmits the latitude/longitude information to the host 300. The alarm device 120 sends a warning signal controlled by the host 300. The bio-signal sensor 30 detects a body information of an organism and transmits the body information to the host 300. The recording device 200 stores the data and information of the host 300. The emergency button 10 controls the host 300 to transmit a rescue information to the driving health monitoring center 500. The telephone equipment 20 generates a voice signal to the host 300. The high-voltage defibrillator 100 transmits signals to the organism controlled by the host 300.

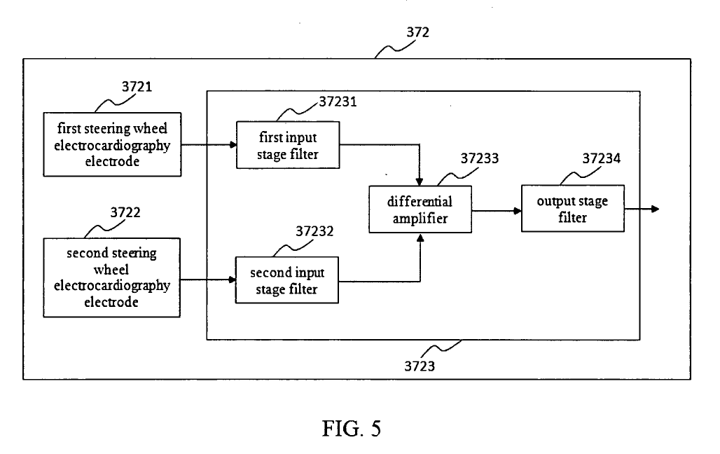

The host 300 is an automobile computer system or portable computer system. The wireless transmission system 400 is a third generation mobile communication system or a general packet radio service system. The alarm device 120 is selected from at least one of a car audio, a light and a screen. The bio-signal sensor 30 is an electrocardiography detecting device 37 for detecting the electrocardiography signal of an organism. The electrocardiography detecting device 37 further comprises a wireless electrocardiography detecting device 371 and wired electrocardiography detecting device 372. Please refer to FIG. 5, which is a schematic drawing of the electrocardiography detecting device according to the present invention. The wired electrocardiography detecting device 372 comprises a first steering wheel electrocardiography electrode 3721, a second steering wheel electrocardiography electrode 3722, and an electrocardiography amplifier 3723, wherein the first steering wheel electrocardiography electrode 3721 is a conductor and disposed at a side of a steering wheel which one palm of the driver can touch, and the second steering wheel electrocardiography electrode 3722 is a conductor and disposed at the other side of the steering wheel which the other palm of the driver can touch. The electrocardiography amplifier 3723 is including a first input stage filter 37231, a second input stage filter 37232, a differential amplifier 37233 and an output stage filter 37234, wherein an input end of the first input stage filter 37231 is electrically connected to the first steering wheel electrocardiography electrode 3721, an input end of the second input stage filter 37232 is electrically connected to the second steering wheel electrocardiography electrode 3722, an input end of the differential amplifier 37233 is electrically connected to an output end of the first input stage filter 37231 and an output end of the second input stage filter 37232, and an output end of the output stage filter 37234 is electrically connected to the host 300.

One user (i.e. the driver or the passenger who needed) holds the wired electrocardiography detecting device 372 on the steering wheel to detect the user's electrocardiography signal and transmit the user's electrocardiography signal to the host 300 immediately so as to analyze the user health. The vehicle speed monitoring device 80, i.e. G sensor with X-Y-Z, disposed on a vehicle monitors the acceleration/deceleration information of the vehicle and transmits the acceleration/deceleration information to the host 300 immediately so as to monitor driving quality and detect the variation of the steering wheel and the gas pedal simultaneously. At the same time, the vehicle position detecting device 90, i.e. the Global Positioning System (GPS), disposed on the vehicle detects the latitude/longitude information of the vehicle and transmits the latitude/longitude information to the host 300. After the host 300 collects all of the data and information, the recording device 200 stores the user's electrocardiography signal, the acceleration/deceleration information and the latitude/longitude information of the vehicle and transmit to the driving health monitoring center 500 by the wireless transmission device 400 so as to analyze whether the user health is fine or not. When pressing the emergency button 10 by hand, the host 300 controls the telephone equipment 20 to communicate with the driving health monitoring center 500 by the wireless transmission device 400, and the driving health monitoring center 500 responds to the host 300 by the wireless transmission device 400 so as to control the alarm device 120 to send a warning signal and control the high-voltage defibrillator 100 for emergency rescue, such as defibrillating by electrical shock.

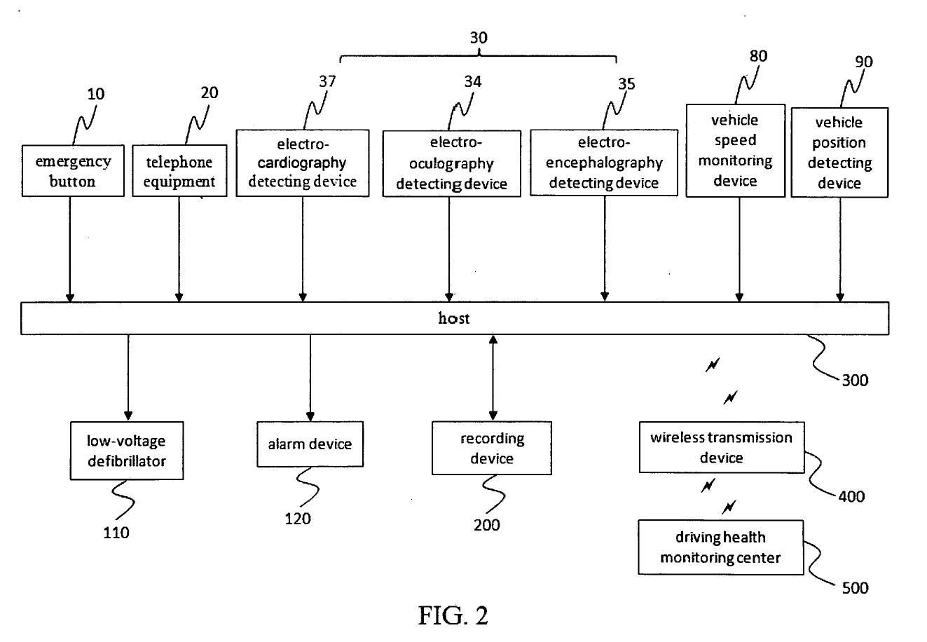

FIG. 2 is a schematic drawing of a sleep detecting and preventing system according to the present invention. The sleep detecting and preventing system 2 comprises a host 300, a driving health monitoring center 500, at least one wireless transmission system 400, at least one vehicle speed monitoring device 80, at least one vehicle position detecting device 90, at least one bio-signal sensor 30, at least one recording device 200, an emergency button 10, a telephone equipment 20, a low-voltage defibrillator 110 and at least one alarm device 120.

The low-voltage defibrillator 110 outputs a control signal to the organism controlled by the host 300. The bio-signal sensor 30 is used for detecting a body information of the organism and transmitting the body information to the host 300 and further comprises an electrocardiography detecting device 37, an electrooculography detecting device 34 and an electroencephalography detecting device 35, wherein the electrooculography detecting device 34 is used for detecting an electrooculography signal of the organism, and the electroencephalography detecting device 35 is used for detecting an electroencephalography signal of the organism.

The host 300, the driving health monitoring center 500, the wireless transmission system 400, the vehicle speed monitoring device 80, the vehicle position detecting device 90, the recording device 200, the emergency button 10, the telephone equipment 20, and the alarm device 120 are the same as above mentioned.

The user (i.e. the driver or the passenger who needed) uses the electrocardiography detecting device 37 to detect user's electrocardiography signal and wears the electrooculography detecting device 34 and the electroencephalography detecting device 35 with the eyeglasses-type or other hat-type to respectively detect user's electrooculography signal and electroencephalography signal, and transmit them to the host 300 immediately so as to analyze the user's sleeping status. The vehicle speed monitoring device 80 disposed on the vehicle monitors the acceleration/deceleration information of the vehicle and transmits the acceleration/deceleration information to the host 300 immediately so as to monitor driving quality and detect the variation of the steering wheel and the gas pedal simultaneously and judge whether the driver is sleeping or has slept. The vehicle position detecting device 90 disposed on the vehicle detects the latitude/longitude information of the vehicle and transmits the latitude/longitude information to the host 300. After the host 300 collects all of the data and information, the recording device 200 stores the user's electrocardiography signal, the electrooculography signal, the electroencephalography signal, the acceleration/deceleration information and the latitude/longitude information of the vehicle and transmit to the driving health monitoring center 500 by the wireless transmission device 400 so as to analyze whether the user is sleeping or has slept. When pressing the emergency button 10 by hand, the host 300 controls the telephone equipment 20 to communicate with the driving health monitoring center 500 by the wireless transmission device 400, and the driving health monitoring center 500 responds to the host 300 by the wireless transmission device 400 so as to control the alarm device 120 to alert a warning signal and control the low-voltage defibrillator 110 to output a low voltage for exciting the organism to freshen up, such as wearing an exciter with sunglasses structure which has a low-voltage electrode.

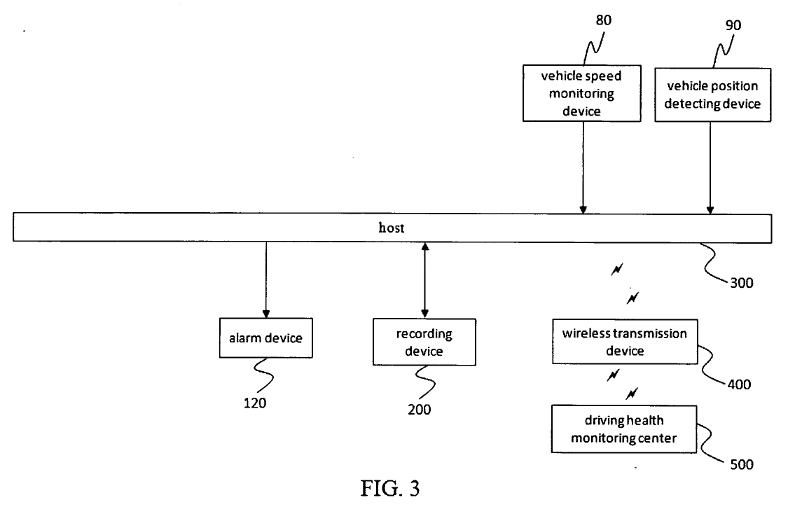

FIG. 3 is a schematic drawing of a driving quality monitoring system according to the present invention. The driving quality monitoring system 3 comprises a host 300, a driving health monitoring center 500, at least one wireless transmission system 400, at least one vehicle speed monitoring device 80, at least one vehicle position detecting device 90, at least one recording device 200 and at least one alarm device 120.

The host 300, the driving health monitoring center 500, the wireless transmission system 400, the vehicle speed monitoring device 80, the vehicle position detecting device 90, the recording device 200 and the alarm device 120 are the same as above mentioned.

The vehicle speed monitoring device 80 disposed on the vehicle monitors the acceleration/deceleration information of the vehicle and transmits the acceleration/deceleration information to the host 300 immediately so as to monitor driving quality and detect the variation of the steering wheel and the gas pedal simultaneously. At the same time, the vehicle position detecting device 90 disposed on the vehicle detects the latitude/longitude information of the vehicle and transmits the latitude/longitude information to the host 300. After the host 300 collects all of the data and information, the host 300 controls the alarm device 120 to alert a warning signal to the driver so as to be a guidance of comfort quality for the passenger, and the recording device 200 stores the acceleration/deceleration information and the latitude/longitude information, and transmits them to the driving health monitoring center 500 by the wireless transmission device 400 so as to manage whether the driver has the driving quality problem.

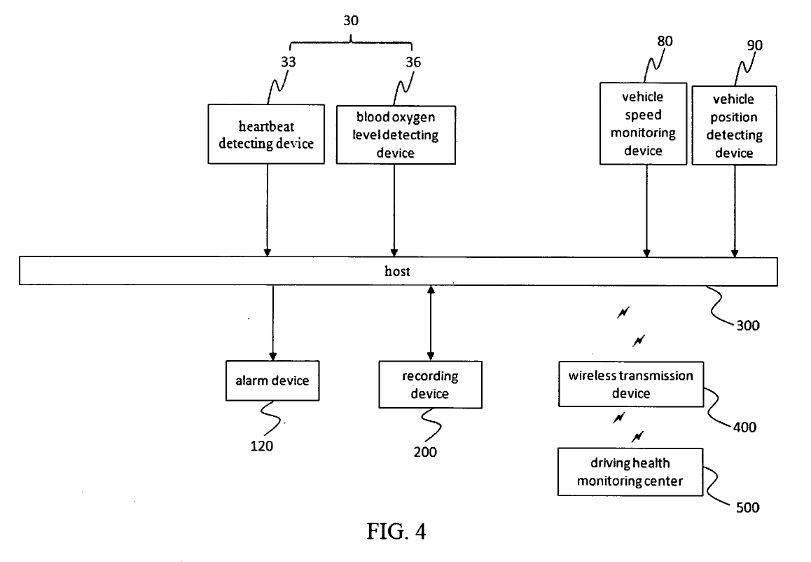

FIG. 4 is a schematic drawing of a blood oxygen and heartbeat detecting system according to the present invention. The blood oxygen and heartbeat detecting system 4 comprises a host 300, a driving health monitoring center 500, at least one wireless transmission system 400, at least one vehicle speed monitoring device 80, at least one vehicle position detecting device 90, at least one bio-signal sensor 30, at least one recording device 200 and at least one alarm device 120.

The bio-signal sensor 30 is used for detecting and transmitting a bio-signal to the host 300 and further comprising a heartbeat detecting device 33 and a blood oxygen level detecting device 36, wherein the heartbeat detecting device 33 is used for detecting a heartbeat signal of the organism and the blood oxygen level detecting device 36 is used for detecting a blood oxygen level signal of the organism.

The host 300, the driving health monitoring center 500, the wireless transmission system 400, the vehicle speed monitoring device 80, the vehicle position detecting device 90, the recording device 200 and the alarm device 120 are the same as above mentioned.

The user (i.e. the driver or the passenger who needed) uses the heartbeat detecting device 33 to detect user's heartbeat signal and uses the blood oxygen level detecting device 36 to detect user's blood oxygen level signal, and transmits them to the host 300 so as to analyze the user's health status. The vehicle speed monitoring device 80 disposed on the vehicle monitors the acceleration/deceleration information of the vehicle and transmits the acceleration/deceleration information to the host 300 immediately so as to monitor driving quality and detect the variation of the steering wheel and the gas pedal simultaneously. At the same time, the vehicle position detecting device 90 disposed on the vehicle detects the latitude/longitude information of the vehicle and transmits the latitude/longitude information to the host 300. After the host 300 collects all of the data and information, the recording device 200 stores the user's heartbeat signal, the user's blood oxygen level signal, the acceleration/deceleration information and the latitude/longitude information of the vehicle and transmit to the driving health monitoring center 500 by the wireless transmission device 400 so as to analyze whether the user has health problem, and the driving health monitoring center 500 responds to the host 300 by the wireless transmission device 400 so as to control the alarm device 120 to alert a warning signal.

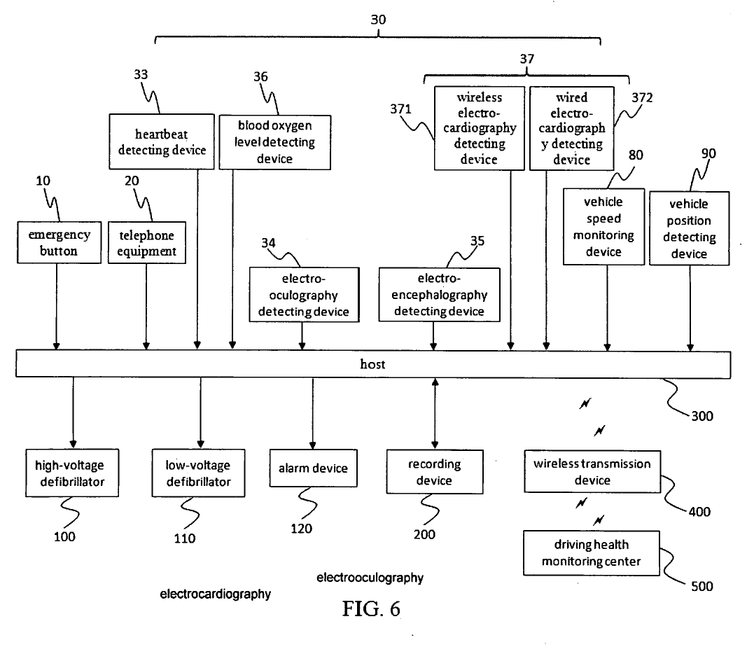

FIG. 6 is a schematic drawing of a system for health management used in vehicle according to the present invention. The system comprises a host 300, a driving health monitoring center 500, at least one wireless transmission system 400, at least one vehicle speed monitoring device 80, at least one vehicle position detecting device 90, at least one bio-signal sensor 30, at least one recording device 200, an emergency button 10, a telephone equipment 20, a high-voltage defibrillator 100, a low-voltage defibrillator 110 and at least one alarm device 120.

The bio-signal sensor 30 is used for detecting and transmitting a bio-signal to the host 300 and comprising a heartbeat detecting device 33, an electrooculography detecting device 34, an electroencephalography detecting device 35, a blood oxygen level detecting device 36, and an electrocardiography detecting device 37, wherein the electrocardiography detecting device 37 further comprises a wireless electrocardiography detecting device 371 and wired electrocardiography detecting device 372.

The host 300, the driving health monitoring center 500, the wireless transmission system 400, the vehicle speed monitoring device 80, the vehicle position detecting device 90, the recording device 200, the emergency button 10, the telephone equipment 20, the high-voltage defibrillator 100, the low-voltage defibrillator 110 and the alarm device 120 are the same as above mentioned.

One user (i.e. the driver or the passenger who needed) holds the wired electrocardiography detecting device 372 on the steering wheel to detect the user's electrocardiography signal, uses the heartbeat detecting device 33 to detect the user's heartbeat signal, uses the blood oxygen level detecting device 36 to detect the user's blood oxygen level signal, wears the electrooculography detecting device 34 and the electroencephalography detecting device 35 with the eyeglasses-type or other hat-type to respectively detect user's electrooculography signal and electroencephalography signal, and transmit them to the host 300 immediately so as to analyze the user's health and sleeping status. The vehicle speed monitoring device 80 disposed on a vehicle monitors the acceleration/deceleration information of the vehicle and transmits the acceleration/deceleration information to the host 300 immediately so as to monitor driving quality and detect the variation of the steering wheel and the gas pedal simultaneously and judge whether the driver is sleeping or has slept. At the same time, the vehicle position detecting device 90 disposed on the vehicle detects the latitude/longitude information of the vehicle and transmits the latitude/longitude information to the host 300. After the host 300 collects all of the data and information, the recording device 200 stores the user's electrocardiography signal, the electrooculography signal, the electroencephalography signal, the heartbeat signal, the blood oxygen level signal, the acceleration/deceleration information and the latitude/longitude information of the vehicle and transmit to the driving health monitoring center 500 by the wireless transmission device 400 so as to analyze whether the user has sleeping or slept problem. When pressing the emergency button 10 by hand, the host 300 controls the telephone equipment 20 inside the vehicle to communicate with the driving health monitoring center 500 by the wireless transmission device 400, and the driving health monitoring center 500 responds to the host 300 by the wireless transmission device 400 so as to control the alarm device 120 to alert a warning signal and control the low-voltage defibrillator 110 to output a low voltage for exciting the driver to freshen up, such as wearing an exciter with sunglasses structure which has a low-voltage electrode, or control the high-voltage defibrillator 100 for emergency rescue, such as defibrillating by electrical shock.

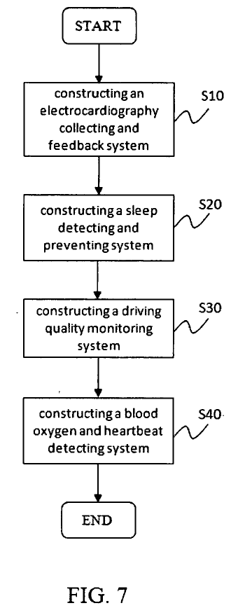

FIG. 7 is a process schematic drawing of a method for health management used in vehicle according to the present invention. The steps of the method comprise:
Step S10: constructing an electrocardiography collecting and feedback system 1 shown as FIG. 1;
Step S20: constructing a sleep detecting and preventing system 2 shown as FIG. 2;
Step S30: constructing a driving quality monitoring system 3 shown as FIG. 3; and
Step S40: constructing a blood oxygen and heartbeat detecting system 4 shown as FIG. 4.

Although the invention has been explained in relation to its preferred embodiment, it is not used to limit the invention. It is to be understood that many other possible modifications and variations can be made by those skilled in the art without departing from the spirit and scope of the invention as hereinafter claimed.

1. A system for health management used in vehicle, comprising:
A) an electrocardiography collecting and feedback system (1);
B) a sleep detecting and preventing system (2);
C) a driving quality monitoring system (3); and
D) a blood oxygen and heartbeat detecting system (4);
wherein the electrocardiography collecting and feedback system (1), the sleep detecting and preventing system (2), the driving quality monitoring system (3) and the blood oxygen and heartbeat detecting system (4) are including:
a) a host (300);
b) a driving health monitoring center (500) processing and responding a data of the host (300);
c) at least one wireless transmission system (400) transmitting a data between the host (300) and the driving health monitoring system (500);
d) at least one vehicle speed monitoring device (80) detecting and transmitting a data of vehicle's velocity to the host (300);
e) at least one vehicle position detecting device (90) detecting and transmitting a data of vehicle's latitude and longitude to the host (300);
f) at least one alarm device (120) sending a warning signal controlled by the host (300);
g) at least one bio-signal sensor (30) detecting and transmitting a bio-signal to the host (300); and
h) at least one recording device (200) storing the data of the host (300).

2. The system for health management used in vehicle as claimed in claim 1, wherein the host (300) is an automobile computer system or a portable computer system.

3. The system for health management used in vehicle as claimed in claim 1, wherein the wireless transmission system (400) is a third generation mobile communication system or a general packet radio service system.

4. The system for health management used in vehicle as claimed in claim 1, wherein the alarm device (120) is selected from at least one of a car audio, a light and a screen.

5. The system for health management used in vehicle as claimed in claim 1, wherein the bio-signal sensor (30) of the electrocardiography collecting and feedback system (1) is an electrocardiography detecting device (37).

6. The system for health management used in vehicle as claimed in claim 1, wherein the electrocardiography collecting and feedback system (1) is further comprising an emergency button (10), a telephone equipment (20) and a high-voltage defibrillator (100), wherein the emergency button (10) is used for controlling the host (300) to transmit a rescue information to the driving health monitoring center (500), the telephone equipment (20) is used for producing a voice signal to the host (300), and the high-voltage defibrillator (100) is outputting a signal to an organism controlled by the host (300).

7. The system for health management used in vehicle as claimed in claim 1, wherein the bio-signal sensor (30) of the sleep detecting and preventing system (2) is selected from at least one of an electrocardiography detecting device (37), an electrooculography detecting device (34) and an electroencephalography detecting device (35).

8. The system for health management used in vehicle as claimed in claim 1, wherein the sleep detecting and preventing system (2) further comprising a low-voltage defibrillator (110) outputting a control signal to an organism controlled by the host (300).

9. The system for health management used in vehicle as claimed in claim 6, wherein the sleep detecting and preventing system (2) further comprising a low-voltage defibrillator (110) outputting a control signal to an organism controlled by the host (300).

10. The system for health management used in vehicle as claimed in claim 1, wherein the blood oxygen and heartbeat detecting system (4) further comprising a blood oxygen level detecting device (36) and a heartbeat detecting device (33).

11. The system for health management used in vehicle as claimed in claim 5 or 7, wherein the electrocardiography detecting device (37) is a wireless electrocardiography detecting device (371) or wired electrocardiography detecting device (372).

12. The system for health management used in vehicle as claimed in claim 11, wherein the wired electrocardiography detecting device (372) further comprising:
A) a first steering wheel electrocardiography electrode (3721) disposed at a side of a steering wheel which one palm of the driver can touch and the first steering wheel electrocardiography electrode (3721) is a conductor;
B) a second steering wheel electrocardiography electrode (3722) disposed at the other side of the steering wheel which the other palm of the driver can touch and the second steering wheel electrocardiography electrode (3721) is a conductor;
C) an electrocardiography amplifier (3723) including a first input stage filter (37231), a second input stage filter (37232), a differential amplifier (37233) and an output stage filter (37234), wherein an input end of the first input stage filter (37231) is electrically connected to the first steering wheel electrocardiography electrode (3721), an input end of the second input stage filter (37232) is electrically connected to the second steering wheel electrocardiography electrode (3722), an input end of the differential amplifier (37233) is electrically connected to an output end of the first input stage filter (37231) and an output end of the second input stage filter (37232), and an output end of the output stage filter (37234) is electrically connected to the host (300).

13. A method for health management used in vehicle, comprising:
A) constructing an electrocardiography collecting and feedback system (1);
B) constructing a sleep detecting and preventing system (2);
C) constructing a driving quality monitoring system (3); and
D) constructing a blood oxygen and heartbeat detecting system (4);
wherein the electrocardiography collecting and feedback system (1), the sleep detecting and preventing system (2), the driving quality monitoring system (3) and the blood oxygen and heartbeat detecting system (4) are including:
a) a host (300);
b) a driving health monitoring center (500) processing and responding a data of the host;
c) at least one wireless transmission system (400) transmitting a data between the host (300) and the driving health monitoring system (500);
d) at least one vehicle speed monitoring device (80) detecting and transmitting a data of vehicle's velocity to the host (300);
e) at least one vehicle position detecting device (90) detecting and transmitting a data of vehicle's latitude and longitude to the host (300);
f) at least one alarm device (120) sending a warning signal controlled by the host (300);
g) at least one bio-signal sensor (30) detecting and transmitting a bio-signal to the host (300); and
h) at least one recording device (200) storing the data of the host (300).

14. The method for health management used in vehicle as claimed in claim 13, wherein the host (300) is an automobile computer system or a portable computer system.

15. The method for health management used in vehicle as claimed in claim 13, wherein the wireless transmission system (400) is a third generation mobile communication system or a general packet radio service system.

16. The method for health management used in vehicle as claimed in claim 13, wherein the alarm device (120) is selected from at least one of a car audio, a light and a screen.

17. The method for health management used in vehicle as claimed in claim 13, wherein the bio-signal sensor (30) of the electrocardiography collecting and feedback system (1) is an electrocardiography detecting device (37).

18. The method for health management used in vehicle as claimed in claim 13, wherein the electrocardiography collecting and feedback system (1) is further comprising an emergency button (10), a telephone equipment (20) and a high-voltage defibrillator (100), wherein the emergency button (10) is used for controlling the host (300) to transmit a rescue information to the driving health monitoring center (500), the telephone equipment (20) is used for producing a voice signal to the host (300), and the high-voltage defibrillator (100) is outputting a signal to an organism controlled by the host (300).

19. The method for health management used in vehicle as claimed in claim 13, wherein the bio-signal sensor (30) of the sleep detecting and preventing system (2) is selected from at least one of an electrocardiography detecting device (37), an electrooculography detecting device (34) and an electroencephalography detecting device (35).

20. The method for health management used in vehicle as claimed in claim 13, wherein the sleep detecting and preventing system (2) further comprising a low-voltage defibrillator (110) outputting a control signal to an organism controlled by the host (300).

21. The method for health management used in vehicle as claimed in claim 18, wherein the sleep detecting and preventing system (2) further comprising a low-voltage defibrillator (110) outputting a control signal to an organism controlled by the host (300).

22. The method for health management used in vehicle as claimed in claim 13, wherein the blood oxygen and heartbeat detecting system (4) further comprising a blood oxygen level detecting device (36) and a heartbeat detecting device (33).

23. The method for health management used in vehicle as claimed in claim 17 or 19, wherein the electrocardiography detecting device (37) is a wireless electrocardiography detecting device (371) or wired electrocardiography detecting device (372).

24. The method for health management used in vehicle as claimed in claim 23, wherein the wired electrocardiography detecting device (372) further comprising:
A) a first steering wheel electrocardiography electrode (3721) disposed at a side of a steering wheel which one palm of the driver can touch and the first steering wheel electrocardiography electrode (3721) is a conductor;
B) a second steering wheel electrocardiography electrode (3722) disposed at the other side of the steering wheel which the other palm of the driver can touch and the second steering wheel electrocardiography electrode (3721) is a conductor;
C) an electrocardiography amplifier (3723) including a first input stage filter (37231), a second input stage filter (37232), a differential amplifier (37233) and an output stage filter (37234), wherein an input end of the first input stage filter (37231) is electrically connected to the first steering wheel electrocardiography electrode (3721), an input end of the second input stage filter (37232) is electrically connected to the second steering wheel electrocardiography electrode (3722), an input end of the differential amplifier (37233) is electrically connected to an output end of the first input stage filter (37231) and an output end of the second input stage filter (37232), and an output end of the output stage filter (37234) is electrically connected to the host (300).
